(19) United States
(12) Patent Application Publication
(10) Pub. No.: US 2024/0104392 A1
(43) Pub. Date: Mar. 28, 2024

(54) DISTRIBUTED LEARNING-BASED MULTI-TASK VISION TRANSFORMER THROUGH RANDOM PATCH PERMUTATION SUBSTITUTION AND TRANSFORMATION METHOD USING THE SAME

(71) Applicant: Korea Advanced Institute of Science and Technology, Daejeon (KR)

(72) Inventors: JongChul YE, Daejeon (KR); Sangjoon PARK, Daejeon (KR)

(73) Assignee: Korea Advanced Institute of Science and Technology, Daejeon (KR)

(21) Appl. No.: 18/351,945

(22) Filed: Jul. 13, 2023

(30) Foreign Application Priority Data
Sep. 19, 2022 (KR) 1020220117686

(51) Int. Cl. G06N 3/098; G06V 10/774; G06V 10/94

(52) U.S. Cl. CPC G06N 3/098; G06V 10/7747; G06V 10/95

(57) ABSTRACT

Disclosed are a distributed learning-based multi-task vision transformer through random patch permutation and a transformation method using the same. The transformation method using the distributed learning-based multi-task vision transformer through random patch permutation may include preparing, using a task non-specific patch embedder, a patch embedding for each client and passing the patch embedding through a permutation module and then transmitting the patch embedding to a server; and storing ; by the server, the received patch embedding and using the patch embedding to update body and tail parts of a vision transformer model.

### CROSS-REFERENCE TO RELATED APPLICATION(S)

This application claims the priority benefit of Korean Patent Application No 10-2022-0117686, filed on Sep. 19, 2022, in the Korean Intellectual Property Office, the disclosure of which is incorporated herein by reference.

### BACKGROUND

### 1. Field of the Invention

Example embodiments relate to a distributed learning-based multi-task vision transformer through random patch permutation for personal information protection and a transformation method using the same. This study was conducted with the support of the Korea Health Industry Development Institute's Convergence Physician Scientist Training Project funded by the Ministry of Health and Welfare.

2. Description of the Related Art

Artificial intelligence (AI) has been gaining unprecedented popularity due to its potential to revolutionize various fields of data science. In particular, a deep neural network (DNN) has attained expert-level performance in various application fields of medical imaging.

An enormous amount of data is required to enable an AI model to provide precise decision support with robustness. However, data collected from volunteer participation of a few institutions may not fully meet the amount to guarantee robust performance. Even for large public datasets, unquantifiable biases stemming from limited geographic regions and patient demographics, such as ethnicities and races, may be inevitably included, which may cause performance instability in real-world applications. In particular, for a newly emerging disease like coronavirus disease 19 (COVID-19), this limitation may be exacerbated since it is difficult to promptly build a large and well-curated dataset with sufficient diversity.

Therefore, the ability to collaborate between a plurality of institutions is critical for successful application of AI in medical imaging, but rigorous regulations and ethical restrictions for sharing patient data are another obstacle to multi-institutional collaborative work. Several formal regulations and guidelines, such as the United States Health Insurance Portability and Accountability Act (HIPAA) and the European General Data Protection Regulation (GDPR), state strict regulations regarding storage and sharing of patient data.

Accordingly, distributed learning methods that perform learning tasks at edge devices in a distributed fashion may be effectively utilized in healthcare research. Specifically, distributed learning was introduced to enable model training with data that resides on a source device without sharing. Federated learning (FL) is one of methods that enable distributed clients to collaboratively learn a shared model without sharing their training data. However, it still holds several limitations in that it is significantly dependent on client-side computation resources for parallel computation and not completely free from privacy concerns with gradient inversion attack. Another distributed learning method, split learning (SL), which splits a network into parts between a client and a server, is a promising method that puts low computational load at edge devices. However, it has a disadvantage in that communication overhead between the client and the server is high and also has limitations in privacy preservation since private data may be restored by a malicious attack with feature hijacking and model inversion. In addition, split learning (SL) shows significantly slower convergence compared with federated learning (FL) and does not show optimal performance under significantly skewed data distribution between clients.

Inspired by a modular decomposition structure of a vision transformer (ViT), a novel distributed learning method called Federated Split Task-Agnostic (FESTA) learning was recently proposed for distributed multi-task collaboration using a vision transformer (ViT) architecture. A FESTA framework, equipped with a shared task-agnostic vision transformer (ViT) body on a server-side and multiple task-specific convolutional neural network (CNN) heads and tails on a clients-side, was able to balance the merit of FL and SL, thereby improving the performance of individual tasks under distributed multi-task collaboration setting at a level even better than a single-task expert model trained in a data-centralized manner.

Nevertheless, there are some critical limitations with the FESTA framework. First, since the model needs to continuously share features and gradients as well as head and tail parts of the network, communication overhead is higher than that of SL and FL, which may make practical implementation difficult. Second, it was found that large size head and tail parts in the original FESTA tend to reduce the role of the shared body, resulting in a small improvement compared to single task learning despite the vision transformer (ViT)'s potential for multi-task learning (MTL). Also, since features transmitted to a server body may be hijacked and reverted to original data by an outside malicious attacker or an “honest-but-curious” server in the same manner in SL, the FESTA framework was not free from a privacy issue.

Non-patent document 1 is as follows: H. Chen, Y. Wang, T. Guo, C. Xu, Y. Deng, Z. Liu, S. Ma, C. Xu, C. Xu, and W. Gao, “Pre-trained image processing transformer,” arXiv preprint arXiv:2012.00364, 2020.

### SUMMARY

Example embodiments describe a distributed learning-based multi-task vision transformer through random patch permutation and a transformation method using the same and more particularly, provide technology for adopting a simple patch embedder that performs random permutation and improving multi-task learning performing without sacrificing privacy.

Example embodiments provide a distributed learning-based multi-task vision transformer through random patch permutation that may protect personal information, which was infeasible in the existing method, and at the same time, may reduce a communication amount to about a half by integrating a distributed learning method and a multi-task learning using a vision transformer model and, at the same time by applying technology called a random patch permutation module, and a transformation method using the same.

According to an example embodiment, there is provided a transformation method using a distributed learning-based multi-task vision transformer through random patch permutation, the transformation method including preparing, using a task non-specific patch embedder, a patch embedding for each client and passing the patch embedding through a permutation module and then transmitting the patch embedding to a server; and storing, by the server, the received patch embedding and using the patch embedding to update body and tail parts of a vision transformer model.

The vision transformer model that performs multi-task learning may be separated into a head and tail part that is a model of a client side and a body part that is a model of a server side and learning may be performed with a distributed learning method without directly sharing data.

The preparing and the passing and then transmitting may include randomly shuffling, using the permutation module, patch permutation before transmitting patch features from a client side to the server and transmitting the same.

The permutation module may be a random patch permutation module and configured to randomly patch-permutate data transmitted from a client side to a server side to transmit representational feature data in which original data is unidentifiable.

The permutation module may be a random patch permutation module and configured to allow only some of model weights aggregated and distributed by a server side to be shared to make it infeasible to restore the entire data in reverse order.

The transformation method may further include performing, by the body part of the vision transformer model of the server, forward pass with permutated patch features and transmitting encoded features back to the client.

The transformation method may further include reversing, by the client, permutation with a stored key, transmitting reverted features to a task-specific tail part, and yielding a final output. The preparing and the passing and then transmitting may include randomly shuffling, using the permutation module, patch permutation before transmitting patch features from a client side to the server and storing the key to reverse the permutation on the client side.

The transmitting the encoded features back to the client may include performing, using the permutation module, back-propagation in order of tail, body, and head of the vision transformer model that is the opposite way of forward propagation.

According to another example embodiment, there is provided a transformation method using a distributed learning-based multi-task vision transformer through random patch permutation, the transformation method including randomly shuffling, using a permutation module, patch permutation and storing a key to reverse permutation on a client side and then transmitting patch features from a client to a server; performing, by a body part of a vision transformer model of the server, forward pass with permutated patch features and transmitting encoded features back to the client; and reversing, by the client, the permutation with the stored key, passing reverted features to a task-specific tail part, and yielding a final output.

The transmitting the encoded features back to the client may include performing, using the permutation module, back-propagation in order of tail, body, and head of the vision transformer model that is the opposite way of forward propagation.

The transmitting the patch features to the server may include preparing, using a task non-specific patch embedder, a patch embedding for each client and passing the patch embedding through the permutation module and then transmitting the patch embedding to the server.

The transformation method may further include storing, by the server, the received patch embedding and using the patch embedding to update body and tail parts of a vision transformer model.

According to still another example embodiment, there is provided a distributed learning-based multi-task vision transformer through random patch permutation, the transformer including a head part configured to prepare, using a task non-specific patch embedder, a patch embedding for each client, to pass the patch embedding through a permutation module and then transmit the patch embedding to a server; and a feature storage configured to store the received patch embedding in the server and to use the patch embedding to update and tail parts of a vision transformer model.

The vision transformer model that performs multi-task learning may be separated into a head and tail part that is a model of a client side and a body part that is a model of a server side and learning may be performed with a distributed learning method without directly sharing data.

The head part may be configured to randomly shuffle, using the permutation module, patch permutation before transmitting patch features from a client side to the server and to transmit the same.

The permutation module may be a random patch permutation module and configured to randomly patch-permutate data transmitted from a client side to the server side to transmit representational feature data in which original data is unidentifiable.

The permutation module may be a random patch permutation module and configured to allow only some of model weights aggregated and distributed by the server side to be shared to make it infeasible to restore the entire data in reverse order.

The transformer may further include a body part configured to perform forward pass with permutated patch features in the body part of the vision transformer model of the server and to transmit encoded features back to the client.

The transformer may further include a tail part configured to reverse, by the client, permutation with a stored key, to transmit everted features to a task-specific tail part, and to a final output. The head part may be configured to randomly shuffle, using the permutation module, patch permutation before transmitting patch features from a client side to the server and to store the key to reverse the permutation on the client side.

The body part may be configured to perform, using the permutation module, back-propagation order of tail, body, and head of the vision transformer model that is the opposite way of forward propagation.

According to some example embodiments, it is possible to provide a distributed learning-based multi-task vision transformer through random patch permutation that may protect personal information, which was infeasible in the existing method, and at the same time, may reduce a communication amount to about a half by integrating a distributed learning method and a multi-task learning using a vision transformer model and, at the same time, by applying technology called a random patch permutation module, and a transformation method using the same.

Also, according to some example embodiments, it is possible to effectively disseminate a platform through effective personal information protection compared to an existing distributed learning method.

Also, according to some example embodiments, it is possible to improve performance of individual task-specific artificial intelligence (AI) models through wide dissemination of a multi-task distributed learning platform and application to various modalities.

Also, according to some example embodiments, it is possible to reduce a communication amount, cost, and time in a distributed learning process through efficient server-client communication.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 illustrates a structure of existing Federated Split Task-Agnostic (FESTA);

FIG. 2 illustrates a learning method of existing FESTA;

FIG. 3 illustrates a structure of a distributed learning-based multi-task vision transformer through random patch permutation according to an example embodiment;

FIG. 4 illustrates a learning method of a distributed learning-based multi-task vision transformer through random patch permutation according to an example embodiment;

FIG. 5 illustrates a permutation module to enhance privacy according to an example embodiment;

FIG. 6 illustrate different permutation patterns for each data of each client according to an example embodiment;

FIG. 7 is a flowchart illustrating a transformation method using a distributed learning-based multi-task vision transformer through random patch permutation according to an example embodiment;

FIG. 8 is a diagram illustrating a distributed learning-based multi-task vision transformer through random patch permutation according to an example embodiment; and

FIG. 9 is a flowchart illustrating a transformation method using a distributed learning-based multi-task vision transformer through random patch permutation according to another example embodiment.

### DETAILED DESCRIPTION

Hereinafter, example embodiments will be described with reference to the accompanying drawings. However, various modifications may be made to the example embodiments and the scope of the present invention is not limited to the following example embodiments. Also, some example embodiments are provided to fully explain the present invention to one of ordinary skill in the art. Shapes and sizes of components in the drawings may be exaggerated for clearer explanation.

As artificial intelligence (AI) models show excellent performance in the fields of medical imaging, various related products are being released. However, the models are trained only for some data in a centralized learning manner and, in many cases, do not show excellent generalization performance and require multiple data to secure the excellent generalization performance. Accordingly, although distributed learning methods, such as federated learning (FL) have been developed, there is still a risk that personal information may be restored due to incomplete protection of the personal information and practical application is difficult due to a large communication amount. Also, existing technology that enables performance improvement using multi-learning in a distributed learning method has limitations in that a communication amount particularly increases.

The following example embodiments provide a method that may solve a privacy invasion issue through restoration of personal information by an attacker and a server, which is an issue in existing distributed learning methods, may maximize performance improvement through a multi-learning framework by maximizing advantages of a multi-learning method, and may also reduce a communication amount between a server and a client.

The example embodiments protect personal information by randomly shuffling and transmitting patch permutation before transmitting representational features from a client side to a server using the intrinsic property of a vision transformer model called patch permutation invariance. In the case of using this method, there is no risk of leakage although representational features are stored in the server and thus, it is possible to reduce a communication amount by initially storing all the representational features and then proceeding with learning. At the same time, by separating the vision transformer model into head, transformer (body), and tail, and sharing the transformer itself, it is possible to improve performance compared to individual tasks using advantages of a multi-learning method.

According to example embodiments, effective dissemination of a platform through effective personal information protection may be expected compared to a existing distributed learning method. As a result, it is expected to improve performance of individual task-specific AI models through wide dissemination of a multi-task distributed learning platform and application to various modalities. At the same time, it is expected to reduce a communication amount, cost, and time in an actual distributed learning process through efficient server-client communication.

In the example embodiment, proposed is a multi-task distributed learning using a vision transformer (ViT) with random patch permutation. Instead of using a convolutional neural network (CNN)-based head as in Federated Split Task-Agnostic (FESTA), p-FESTA according to the example embodiment may improve multi-task learning performance without sacrificing privacy by adopting a simple patch embedder that performs random permutation. Experimental results confirm that the proposed method significantly improves advantages of multi-task collaboration, communication efficiency, and personal information protection, illuminating actual multi-task distributed learning in the fields of medical imaging.

In detail, the example embodiments enhance efficient multi-task learning (MTL) with privacy protection by introducing a p-FESTA framework that is FESTA learning of permutating a pure vision transformer (ViT). Although the overall composition of p-FESTA is similar to that of FESTA, instead of using a CNN-based head, p-FESTA adopts a simple and task non-specific patch embedder, such as a vanilla vision transformer (ViT), enforcing self-attention within a transformer architecture to improve MTL performance. For privacy protection, introduced is a permutation module that randomly shuffles the order of all patch features to prevent an outside attacker or an “honest but curious” server from reverting features into original data containing personal information.

The new architectural change provides several unique advantages to p-FESTA. First, communication overhead is significantly reduced by storing features to be used throughout the entire learning process. Also, the benefit of MTL is enhanced by enforcing a head to play a small role and a multi-task body to perform heavy lifting. Also, data privacy protection is also enhanced with a simple but effective permutation module using the intrinsic property of a vision transformer (ViT).

Initially, the vision transformer (ViT) is described.

The vision transformer (ViT), a recently introduced deep learning model equipped with an exquisite attention mechanism inspired by its successful application in natural language processing (NLP), has demonstrated impressive performance across many vision tasks. Multi-head self-attention in the vision transformer (ViT) may flexibly attend to a sequence of image patches to encode a cue, enabling the model to be robust to nuisances like occlusion, spatial permutation, and adversarial perturbation, such that the model may be more shape-biased like human than a CNN-based model.

In addition, a modular design of the vision transformer (ViT) is simple, implying that components may be easily decomposed into parts: a head configured to project image patches into embeddings, a transformer body configured to encode the embeddings, and a tail configured to yield a task-specific output. This easily decomposable design offers the possibility in the application for MTL. Attention needs to be paid to that motivation of MTL is originated from attempts to mitigate a data insufficiency problem in which the number of data for an individual task is limited. MTL may offer an advantage of improving data efficiency, reducing overfitting through shared representation, and faster convergence by leveraging auxiliary knowledge.

In particular, MTL with a transformer-based model has emerged as a popular approach to improve the performance of a closely related task in NLP. In this approach, a shared transformer learns several related tasks simultaneously, like sentence classification and word prediction, and the tasks-specific module yields an outcome for each task. The model trained with an MTL strategy generally shows improved performance in a wide range of tasks. Although study is not well performed as in language, the decomposable design of the vision transformer (ViT) enabled the application of MTL to a visual transformer model. In an early approach (non-patent document 1), the vision transformer (ViT) was divided into task-specific head, tail, and shared transformer structures across tasks, and it was possible to attain similar generalization performance with fewer training stages by sharing the transformer model among the related tasks.

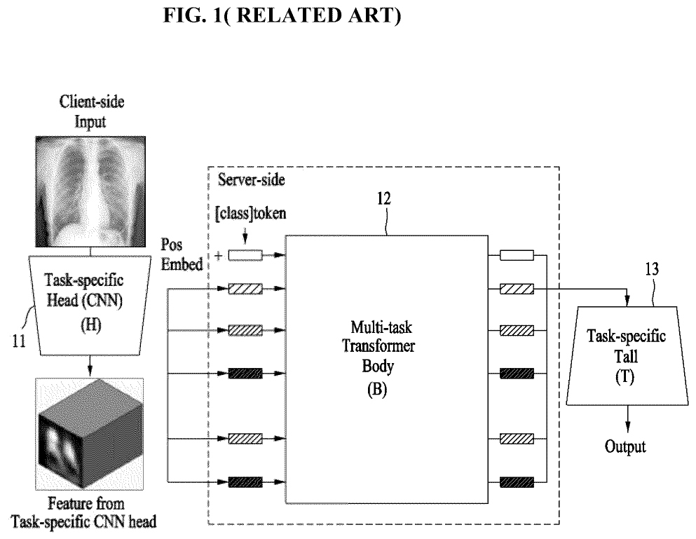

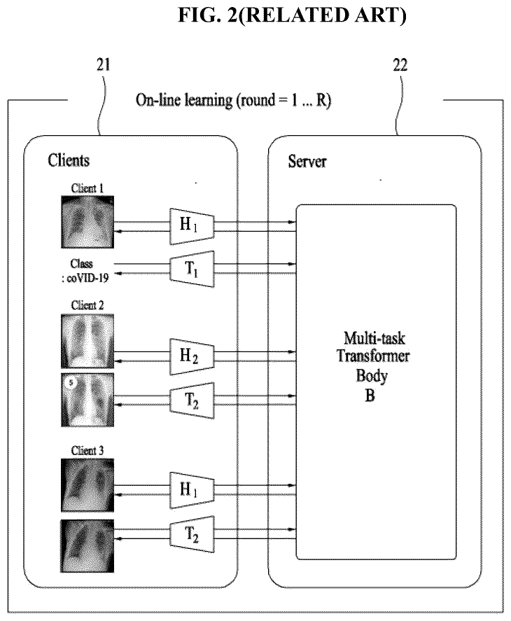

FIG. 1 illustrates a structure of existing FESTA, and FIG. 2 illustrates a learning method of existing FESTA.

Referring to FIGS. 1 and 2, the main motivation of an existing FESTA framework is to provide a framework to maximally use distinct strengths of federated learning (FL) and split learning (SL_ methods and to improve the performance of individual tasks with collaboration between clients that perform various tasks.

Let [symbol] =∪ₖ₌₁^{K} [symbol] ₖ be a group of client sets with different tasks. Here, K denotes the number of tasks and client set Cₖ includes one or more clients having different data sources for a k-th task, that is, [symbol] ₖ={c₁^{k}, c₂^{k}, . . . , c_{N}_{ₖ}^{k}:Nₖ≥1}. Each Client 21 in the client set for the k-th task has its own task-specific model architecture for head H_{c} 11 and tail T_{c} 13. Here, a transformer body B 12 on the side of a server 22 is shared.

For training, the server 22 and each client 21 initialize a weight of each sub-network with random initialization or from pre-trained parameters. For learning round i=1, 2, . . . R, the individual clients 21 perform forward pass on their task-specific head H_{c} using local training data ((x_{c}⁽ⁱ⁾, y_{c}⁽ⁱ⁾))ᵢ₌₁^{N}^{_{c}} and transmit intermediate feature h_{c}⁽ⁱ⁾ to the server 22. h_{c}⁽ⁱ⁾= [symbol] _{c}(x_{c}⁽ⁱ⁾). Then, the transformer body B 12 receives intermediate features from all the clients 21, acquire features b_{c}⁽ⁱ⁾ in parallel with the forward pass, and send the same to each client 21 c. Here, b_{c}⁽ⁱ⁾= [symbol] (b_{c}⁽ⁱ⁾). With the features b_{c}⁽ⁱ⁾, the task-specific tail in the client 21 yields output ŷ_{c}⁽ⁱ⁾= [symbol] _{c}(b_{c}⁽ⁱ⁾) and forward pass is completed. Back-propagation is performed exactly in an opposite way, that is, in order of tail, body, and head. First, loss is computed in tail as [symbol] _{c}(y_{c}⁽ⁱ⁾, [symbol] _{c}( [symbol] ( [symbol] _{c}(x_{c}⁽ⁱ⁾)))). Here, [symbol] _{c}(y, y̅) denotes a c task-specific loss between a target y and estimate y̅. Then, gradients are forwarded from tail, body to head in reverse order using a chain rule.

For multi-task body update, optimization is performed by fixing the head and the tail. For task-specific head and tail update, an optimization problem is solved by fixing the transformer body. Also, per every “UnifyingRounds,” the server 22 aggregates, averages, and distributes head and tail parameters between the clients 21 that participate in the same task, as in FedAvg.

The previous study shows that FESTA along with MTL improves the individual performance of a client in collaboration, while resolving a data governance and ownership issue as well as eliminating the need to transmit huge weights of a transformer body.

The FESTA framework still has several disadvantages. First, communication cost may be higher since features and gradients need to be continuously exchanged between a server and clients as in split learning (SL) but head and tail weights also need to be aggregated between the clients as in federated learning (FL). Accordingly, the total communication cost is inevitably higher than split learning (SL), and even higher than federated learning (FL) depending on a network size. Second, as shown in the disclosed study without the transformer body, CNN head and tail themselves already have strong representation capacity, which may reduce the role of the transformer body between the head and the tail. Third, privacy concerns may arise since there is no privacy protection method from a model inversion attack on a feature transmitted from the client to the server.

The proposed p-FESTA according to an example embodiment is a framework devised to mitigate such disadvantages.

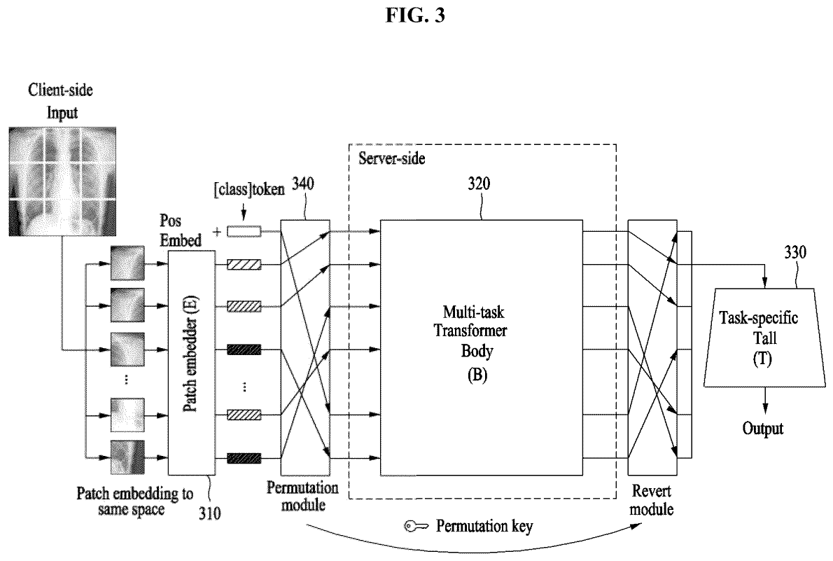

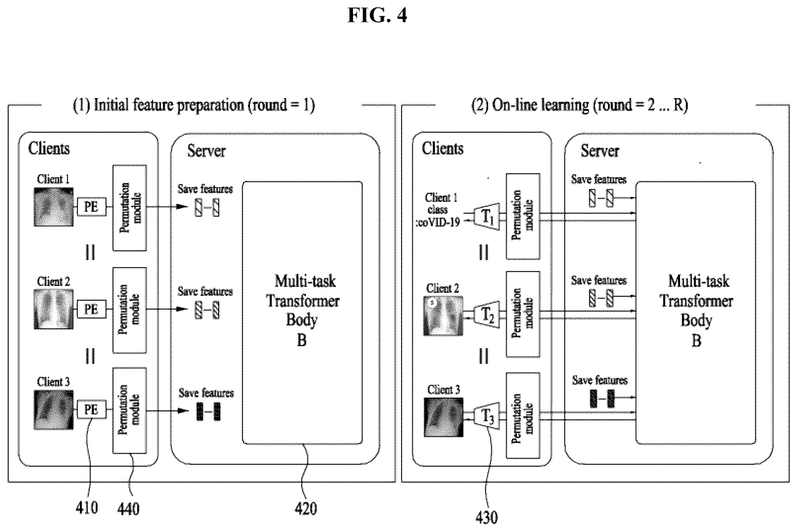

FIG. 3 illustrates a stricture of a distributed learning-based multi-task vision transformer through random patch permutation according to an example embodiment, and FIG. 4 illustrates a learning method of a distributed learning-based multi-task vision transformer through random patch permutation according to an example embodiment.

Referring to FIGS. 3 and 4, the overall composition of p-FESTA is similar to that of FESTA that decomposes a network into a head H 310, 410, a body B 320, 420, and a tail T 330, 430. However, unlike the existing FESTA, a CNN head tailored for each task is not used. If the CNN head is powerful enough to play a major role in a task, it may hinder a shared transformer from being an important component since there remains little room for improvement with this additional module. Instead, according to an example embodiment, self-attention within a transformer architecture may be enforced to perform heavy lift by adopting a simple and task non-specific patch embedder, such as a vanilla vision transformer (ViT).

Here, the use of a patch embedding in the vanilla vision transformer (ViT) may be prone to an outside attacker that attempts to invert a patch embedder to acquire an original image. To solve this, proposed are novel permutation modules 340 and 340 as illustrated in FIGS. 3 and 4 to prevent an outside attacker or an “honest but curious” server from reverting features into original data containing privacy. In particular, the permutation module 340, 440 randomly shuffles the order of all patch features before transmitting the same to a server, and stores a permutation key to reverse the permutation on a client-side. Then, the transformer body B 320, 420 in the server performs forward pass with the permutated patch features and transmits encoded features back to a client. Then, the client reverses the permutation with the stored key and yields a final output by passing the reverted features to the task-specific tail Tₖ 330, 430. Back-propagation is performed in an exact opposite way in which the same permutation module 340, 440 to forward-propagation is utilized.

The availability of the permutation module 340, 440 attributes to an intriguing property of the vision transformer (ViT) that all the components constituting the transformer body B 320, 420, such as multi-head self-attention, feedforward network, and layer normalization are fundamentally “permutation equivariant.” They are processed independently in a patch-based manner and the order of a patch does not affect the outcome. Therefore, the transformer body B 320, 420 may be trained without performance degradation. Also, since the order of patches is completely shuffled, it is infeasible for a malicious attacker to successfully revert an original image. A method of providing, by the permutation module 330, 340, privacy protection from a malicious attacker is further described below.

In the case of federated learning (FL), privacy is improved by the ephemeral and focused nature of federated aggregation, averaging, and distribution of model update, assuming that the model update is considered to be less informative than original data. However, recent studies have thrown doubt to the false sense of security, showing that private data may be uncovered faithfully only with local model update. In detail, given an access to a global model W and a client's model update ΔW, an attacker may optimize a previous input image prior to producing a gradient that matches the client's model update. However, since only a tail part of the entire model is aggregated and distributed by a server to clients, this type of attack is infeasible for the proposed p-FESTA method. For example, for COVID-19 classification, a task-specific tail is a simple linear classifier with which original data with privacy may not be uncovered.

Split learning (SL) protects privacy in a different way. As the name SL suggests, split learning (SL) splits the entire model into a sub-network of a client-side and a sub-network of a server-side and does not transmit models between a server and clients. Instead, features and gradients are transmitted back and forth between the server and the clients, which may be the prey of the malicious attacker.

When the client transmits an intermediate feature f to the server, an attacker may hijack the corresponding feature f and instead of running the remainder of an SL model, three components, an encoder \hat{F}, a decoder G, and a discriminator D, are trained with their own data. The discriminator D is trained to discriminate between the hijacked feature f and feature \hat{ƒ} encoded by the encoder \hat{F}, which allows the feature \hat{ƒ} to be in the same feature space to the feature f. Simultaneously, the decoder G is trained to learn to decode the feature \hat{ƒ} into an image with a minimal error. Then, the well-trained decoder G may also be used to faithfully decode the hijacked feature f to original data.

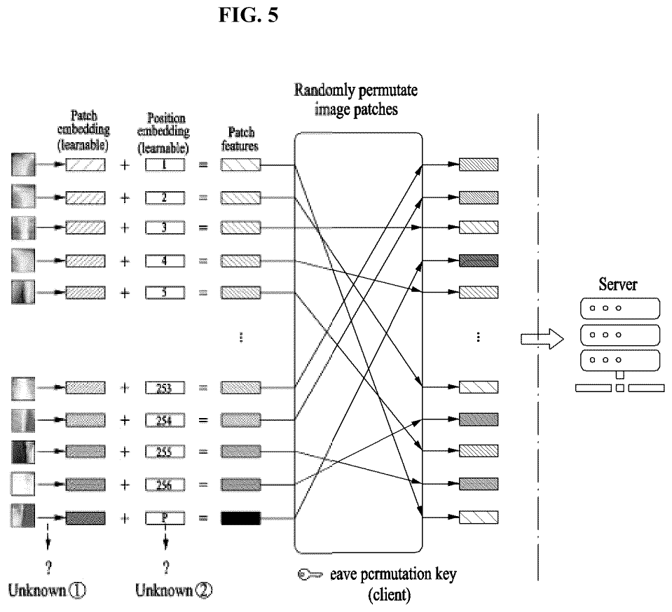

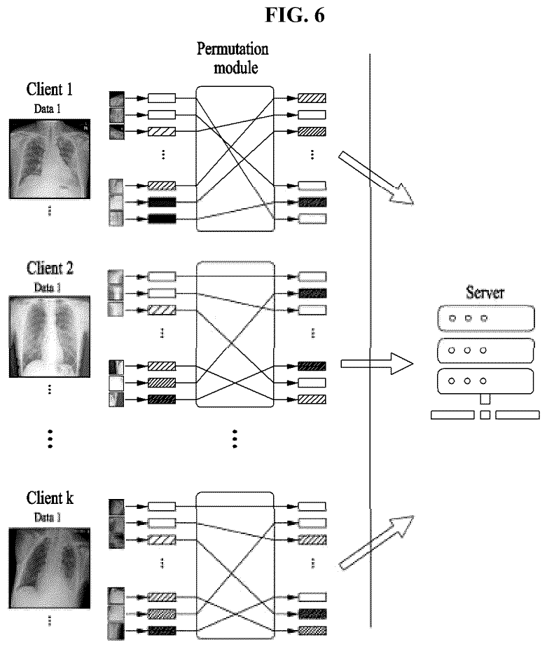

FIG. 5 illustrates a permutation module to enhance privacy according to an example embodiment. Also, FIG. 6 illustrate different permutation patterns for each data of each client according to an example embodiment.

Feature space hijacking is also possible even in p-FESTA according to an example embodiment. A head part of a model according to an example embodiment is relatively simple and may be easy prey for an attacker. Therefore, as illustrated in FIG. 5, a novel permutation module to protect privacy is introduced. The permutation module randomly shuffles the order of all patch features. In an implementation, as illustrated in FIG. 6, permutation of each data of each client is all different without regularity, which may lead to innumerable patterns for all data. With such random permutation, although a malicious attacker or server steals patch features to uncover private data, a position embedding parameter, an unknown learnable variable, may not be inferred since information on the original order of patches is absent. Also, it is infeasible to inverse patch features to image patches since an added position embedding, which is unknown to an attacker, needs to be subtracted first for inversion. This makes a contradiction that the attacker needs to already know one “unknown” to infer the other “unknown,” making the inversion attack a type of underdetermined problem.

Hereinafter, a learning process of p-FESTA is described.

A learning process of p-FESTA is similar to original FESTA, but dissimilar in a plurality of aspects. Instead of a task-specific head Hₖ for each task k, a task non-specific patch embedder H prepares a patch embedding h e for each client c at the beginning, passes the patch embedding h_{c} through a permutation module, and then transmits the patch embedding h_{c} to a server. Then, the server stores the received patch embedding h_{c} on its side and uses the patch embedding h e throughout the remainder of the learning process in order to update body B and tail Tₖ parts of the model. Consequently, since communication to transmit an intermediate feature h_{c} or to update the head H is not required any more, the overall communication cost may be significantly reduced compared to the original FESTA.

The patch embedder that is the head part of the model may not be updated in this configuration. However, fixing parameters of the patch embedder does not cause performance degradation due to a simple structure in which it is possible to embed image patches into the same vector space. Having them trainable rather slightly degrades the performance by causing discrepancy in embedding between tasks. A detailed process of the proposed p-FESTA is described in Algorithm 1 of the following Table 1.

TABLE 1
Algorithm 1: Proposed p-FESTA algorithm

| 1 | Function ServerMain: | | | | |
|---|---|---|---|---|---|
| 2 | Initialize server body weight client head/tail weights | | | | |
| | for tasks k ∈ {1, 2, . . . K} do in parallel | | | | |
| 3 | \| | for clients c ∈ Cₖ do in parallel | | | |
| 4 | \| | \| | h_{c} ← ClientHead(c) | | |
| 5 | └ | └ | Save patch embedding h_{c} in server memory | | |
| 6 | for rounds i = 1, 2, . . . R do | | | | |
| 7 | \| | for tasks k ∈ {1, 2, . . . K} do in parallel | | | |
| 8 | \| | \| | for clients c ∈ Cₖ do in parallel | | |
| 9 | \| | \| | \| | if i = 1 or (i − 1) ∈ UnifyingRounds | |
| | \| | \| | \| | then | |
| 10 | \| | \| | \| | └ | Set client w [symbol] _{_{c}}⁽ⁱ⁾ ← w̅ [symbol] _{,k} |

| 11 | \| | \| | \| | Load h_{c}⁽ⁱ⁾ by batch from server memory |
|---|---|---|---|---|
| | \| | \| | \| | & b_{c}⁽ⁱ⁾ ← [symbol] (h_{c}⁽ⁱ⁾) |
| 12 | \| \| | \| \| | \| \| | $\frac{\partial {L}_{c}^{( i )}}{\partial {b}_{c}^{( i )}} ← ClientTail ( c , {b}_{c}^{( i )} ) \&$ |
| | \| | \| | \| | Backprop. |
| 13 | \| | └ | └ | w_{T}_{_{c}}⁽ⁱ⁺¹⁾ ← ClientUpdate(c) |
| 14 | \| | Update body | | |
| | \| \| \| | ${w}_{ℬ}^{( i + 1 )} ← {w}_{ℬ}^{( i )} - \frac{η}{K} {\sum }_{k = 1}^{K} {\sum }_{c ∈ {C}_{k}} \frac{\partial {L}_{u}^{( i )}}{{N}_{k} S {w}_{𝒮}^{( i )}}$ | | |
| 15 | \| | if i ∈ UnifyingRounds then | | |
| 16 | \| | \| | for tasks k ∈ {1, 2, . . . K} do | |
| 17 | \| \| └ | \| \| └ | \| \| └ | $Update {\overline{w}}_{𝒯 , k} ← \frac{1}{{N}_{k}} {\sum }_{c ϵ {C}_{k}} {w}_{{T}_{c}}^{( i + 1 )}$ |

| | |
|---|---|
| 18 | Function ClientHead (c): |
| 19 | x_{c} ← All data on client c |
| 20 | h_{c} ← [symbol] (x_{c}) |
| 21 | Randomly permutate patch embedding h_{c} |
| 22 | return h_{c} |
| 23 | Function ClientTail (c, b_{c}): |
| 24 | y_{c} ← Current batch of label from client c |
| 25 | L_{c} ← [symbol] _{c}(y_{c}, [symbol] _{c}(b_{c})) & Backprop. |
| 26 | $return \frac{\partial {L}_{c}}{\partial {b}_{c}}$ |
| 27 | Function ClientUpdate (c): |
| 28 | $Backprop . tail , body \& {w}_{{𝒯}_{c}} ← {w}_{{𝒯}_{c}} - η \frac{\partial {L}_{c}}{\partial {w}_{{𝒯}_{c}}}$ |
| 29 | return w [symbol] _{_{c}} |

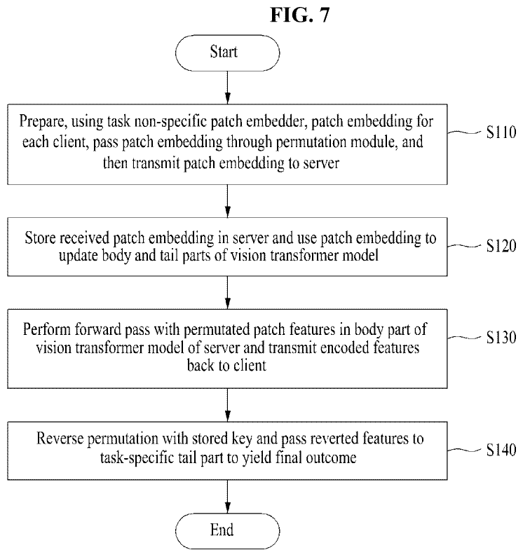

FIG. 7 is a flowchart illustrating a transformation method using a distributed learning-based multi-task vision transformer through random patch permutation according to an example embodiment.

Referring to FIG. 7, the transformation method using the distributed learning-based multi-task vision transformer through random patch permutation according to an example embodiment may include operation S110 of preparing, using a task non-specific patch embedder, a patch embedding for each client, passing the patch embedding through a permutation module, and then transmitting the patch embedding to a server, and operation S120 of storing, by the server, the received patch embedding and using the patch embedding to update body and tail parts of a vision transformer model.

Also, the transformation method may further include operation S130 of performing forward pass with permutated patch features in a body part of the vision transformer model of the server and transmitting the encoded feature back to the client.

Also, the transformation method may further include operation S140 of reversing, by the client, permutation with a stored key, passing reverted features to a task-specific tail part, and yielding a final outcome. Here, operation 110 of preparing the patch embedding and passing the patch embedding through the permutation module, and then transmitting the patch embedding to the server may include randomly shuffling, using the permutation module, patch permutation before transmitting patch features from a client side to the server and storing the key to reverse the permutation on the client side

Hereinafter, the transformation method using the distributed learning-based transformer through random patch permutation according to an example embodiment is described.

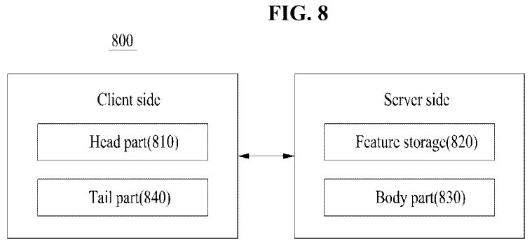

FIG. 8 is a diagram illustrating a distributed learning-based multi-task vision transformer through random patch permutation according to an example embodiment.

Referring to FIG. 8, a distributed learning-based multi-task vision transformer 800 through random patch permutation according to an example embodiment may include a head part 810 and a feature storage 820. Depending on example embodiments, the distributed learning-based multi-task vision transformer 800 through the random patch permutation may further include a body part 830 and a tail part 840. The example embodiments may separate a vision transformer model that performs multi-task learning into a head and tail part that that is a model of a client side and a body part that is a model of a server side and may perform training with a distributed learning method without directly sharing data.

In operation S110, the head part 810 may prepare, using the task non-specific patch embedder, the patch embedding for each client, may pass the patch embedding through the permutation module, and then may transmit the patch embedding to the server. The head part 810 may randomly shuffle patch permutation before transmitting patch features from the client side to the server using the permutation module and then may transmit the same to the server. Here, the head part 810 may randomly shuffle the patch permutation and may store a key to reverse the permutation on a client side.

Here, the permutation module refers to a random patch permutation module and may randomly patch-permutate data transmitted from a client side to a server side to transmit representational feature data in which original data is unidentifiable. Also, the permutation module refers to the random patch permutation module and may allow only some of model weights aggregated and distributed by the server side to be shared to make it infeasible to restore the entire data in reverse order.

In operation S120, the feature storage 820 may store the received patch embedding in the server and may use the patch embedding to update body and tail parts of the vision transformer model.

In operation S130, the body part 830 may perform forward pass with the permutated patch features in the body part of the vision transformer model of the server and may transmit the encoded features back to the client. The body part 830 may perform back-propagation in order of tail, body, and head of the vision transformer model, that is, in the exact opposite way of forward-propagation using the permutation module.

In operation S140, the client may reverse the permutation with the stored key and the tail part 840 may pass the reverted features to the task-specific tail part and may yield the final outcome.

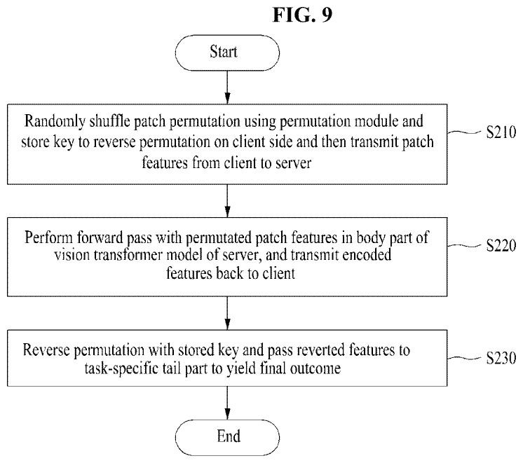

FIG. 9 is a flowchart illustrating a transformation method using a distributed learning-based multi-task vision transformer through random patch permutation according to another example embodiment.

Referring to FIG. 9, the transformation method using the distributed learning-based multi-task vision transformer through random patch permutation according to another example embodiment may include operation S210 of randomly shuffling, using a permutation module, patch permutation and storing a key to reverse permutation on a client side and then transmitting patch features from a client to a server, operation S220 of performing, by a body part of a vision transformer model of the server, forward pass with permutated patch features and transmitting encoded features back to the client, and operation S230 of reversing, by the client, the permutation with the stored key, passing reverted features to a task-specific tail part, and yielding a final output.

The transformation method using the distributed learning-based multi-task vision transformer through random patch permutation according to another example embodiment may be included in or may include a composition of the aforementioned transformation method using the distributed learning-based multi-task vision transformer through random patch permutation according to an example embodiment. Accordingly, repeated description related thereto is omitted.

In operation S210, the head part 810 may randomly shuffle patch permutation using the permutation module and may store the key to reverse the permutation on the client side and then transmit patch features from the client to the server

Meanwhile, the feature storage 820 may store the patch embedding received by the server and may use the patch embedding to update body and tail parts of the vision transformer model.

In operation S220, the body part 830 may perform forward pass with permutated patch features in the body part of the vision transformer model of the server and may transmit the encoded features back to the client. That is, back-propagation may be performed in order of tail, bod, and head of the vision transformer model, that is, in the exact opposite way of forward propagation using the permutation module.

In operation S230, the client may reverse the permutation with the stored key and the tail part 840 may pass the reverted features to the task-specific tail part and may yield the final outcome.

The example embodiments may separate a vision transformer model that performs multi-task learning into a model of a client (accessor) side and a model of a server side and may perform training with a distributed learning method without directly sharing data.

In the above process, representational features and client model weights from the client side need to be transmitted to the server side. In the case of using the existing distributed learning method, if an outside attacker hacks data transmitted to the server in this process or if the server itself uses this data to restore personal information, the data may be restored to a level at which sensitive information is identifiable.

However, the example embodiments additionally apply a random patch permutation module in the above process such that data transmitted to the server side may be randomly patch-permutated to transmit representational feature data in which original data is unidentifiable. Also, the example embodiments may allow only some of model weights aggregated and distributed by the server side to be shared to make it infeasible to restore the entire data in reverse order. Through the above two methods, the example embodiments may further effectively protect personal information more than the existing distributed learning method. The above random patch permutation method utilizes a characteristic of a vision transformer model called a patch permutation invariance.

Also, the example embodiments may improve the performance of a final trained model compared to a single task model by allowing representational features and gradients useful for model training to be shared between clients that perform multi-tasking using advantages of a multi-learning method. Compared to the existing methods, the example embodiments are designed to maximize advantages of the multi-learning method by using a simple head model, by performing learning in the same representation space using the same head between clients, and by allowing a shared transformer model to perform most tasks.

In addition, for a problem of an excessively huge communication amount between a server and a client, which was a problem in the existing multi-task distributed learning method, the example embodiments enable learning with less cost by storing representational feature data on the server side at a start of learning, and by using the same throughout learning, reducing the communication amount to about a half. This is possible since data with personal information protected is transmitted to the server side through a random patch permutation module and also effectively uses the fact that the server may not restore representational features although the representational features are stored on the server side.

The aforementioned personal information protection multi-task distributed learning technology may be applied to various fields as well as medical imaging. Technology for protecting personal information with random permutation using characteristics of a vision transformer model has a wide versatility that it is applicable to any imaging field that allows application of the vision transformer model regardless of modalities and that a task to be performed through an AI model is also applicable to various tasks regardless of a type of the task, such as classification, segmentation, quantification, and the like.

According to example embodiments, it is expected to be most effectively applied in the field of medical imaging in which it is important to protect personal information. In addition to X-ray imaging tested in the study, any “imaging” information may be applied regardless of modalities. Therefore, the proposed technology may be applied in various medical imaging modalities, such as CT, MRI, PET, and the like. Also, due to flexibility of a multi-task learning model, the proposed technology may be effectively applied even in a situation in which each client desires to perform a different task. Also, in addition to the medical imaging field, the proposed technology may be effectively applied even in a distributed learning environment using data of a mobile device, such as a smartphone, since image information stored in a personal smartphone is also data that requires personal information protection.

According to example embodiments, although the proposed technology is developed based on a vision transformer model, it is expected that the proposed technology may be applied to a distributed learning and multi-task learning model in the field of natural language processing in consideration of the fact that a method of processing each image patch in a vision transformer model is almost the same as a method of processing a word in a transformer model for natural language processing. Similar to a method of protecting personal information by randomly shuffling patch permutation, the example embodiments may expect the protection effect for the overall personal information by randomly shuffling order of words even in a natural language model.

As described above, the example embodiments may converge advantages of a federated learning method and a split learning method that are distributed learning methods and, at the same time, improve performance of a vision transformer model through multi-task learning. Also, the example embodiments fundamentally make it infeasible to restore sensitive personal information without using other personal information protection methods by making it infeasible for an attacker to restore sensitive personal information, which was a problem in the existing distributed learning method, through a random patch permutation method. This utilizes the intrinsic property of a vision transformer, that is, patch permutation invariance. Through this, it is possible to reduce the overall communication amount to about a half in a distributed learning process by storing permutated data on a server side and thereby performing learning.

Accordingly, compared to the existing distributed learning method, it is possible to increase a width of performance improvement and to strengthen personal information protection through the multi-learning method and, at the same time, to reduce a communication amount to about a half. Such improvement may be acquired by maximally using the intrinsic property of the vision transformer. Therefore, the example embodiments may provide improved personal information protection compared to the existing patented methods and may be widely applied to medical imaging AI and other imaging AI fields.

The medical imaging AI field in which the technology according to example embodiments may be initially utilized may include a field in which a market size is rapidly growing in recent years and products using an AI model for various modalities, such as X-ray, CT, and MRI, are being launched. However, since such existing products are developed by centrally collecting and learning data for a single task, it is difficult to release a product for a detailed task for which it is difficult to collect a sufficient amount of data. Also, since it is a model trained using data of a small number of institutions, corresponding products may not show excellent generalization performance in many cases. Example embodiments may provide, as technology for eliminating such disadvantages found in the existing market products, a learning framework that may protect personal information of a patient while training a model without directly sharing and centrally collecting data required for product development, and may further improve the performance through wide applications regardless of a task type and using advantages of multi-task learning. Therefore, the proposed technology may be usefully used in companies that develop medical AI models and may be sold by providing development platform products to such corresponding companies. Also, since the proposed is usefully available for research purpose, platform sales may be expected for research in research institutes such as hospitals and universities.

The systems or the apparatuses described herein may be implemented using hardware components, software components, and/or a combination of hardware components and software components. For example, the apparatuses and the components described herein may be implemented using one or more general-purpose or special purpose computers, for example, a processor, a controller, an arithmetic logic unit (ALU), a digital signal processor, a microcomputer, a field programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of the processing device is used as singular; however, one skilled in the art will be appreciated that a processing device may include multiple processing elements and/or multiple types of processing elements. For example, a processing device may include multiple processors or a processor and a controller. In addition, different processing configurations are possible, such as parallel processors.

The software may include a computer program, a piece of code, an instruction, or some combinations thereof, for independently or collectively instructing or configuring the processing device to operate as desired. Software and/or data may be embodied in any type of machine, component, physical equipment, virtual equipment, a computer storage medium or device, to be interpreted by the processing device or to provide an instruction or data to the processing device. The software also may be distributed over network coupled computer systems so that the software is stored and executed in a distributed fashion. The software and data may be stored by one or more computer readable storage media.

The methods according to the above-described example embodiments may be configured in a form of program instructions performed through various computer devices and recorded in computer-readable media. The media may include, alone or in combination with program instructions, data files, data structures, and the like. The program instructions stored in the media may be specially designed and configured for the example embodiments or may be known to one skilled in the computer software art and thereby available. Examples of the media include magnetic media such as hard disks, floppy disks, and magnetic tapes; optical media such as CD-ROM and DVDs; magneto-optical media such as floptical disks; and hardware devices that are configured to store program instructions, such as read-only memory (ROM), random access memory (RAM), flash memory, and the like. Examples of the program instruction may include a machine code as produced by a compiler and include a high-language code executable by a computer using an interpreter and the like.

Although the example embodiments are described with reference to some specific example embodiments and accompanying drawings, it will be apparent to one of ordinary skill in the art that various alterations and modifications in form and details may be made in these example embodiments without departing from the spirit and scope of the claims and their equivalents. For example, suitable results may be achieved if the described techniques are performed in different order, and/or if components in a described system, architecture, device, or circuit are combined in a different manner, and/or replaced or supplemented by other components or their equivalents.

Therefore, other implementations, other example embodiments, and equivalents of the claims are to be construed as being included in the claims.

What is clamed is:

1. A transformation method using a distributed learning-based multi-task vision transformer through random patch permutation, the transformation method comprising:
preparing, using a task non-specific patch embedder, a patch embedding for each client and passing the patch embedding through a permutation module and then transmitting the patch embedding to a server; and
storing, by the server, the received patch embedding and using the patch embedding to update body and tail parts of a vision transformer model.

2. The transformation method of claim 1, wherein the vision transformer model that performs multi-task learning is separated into a head and tail part that is a model of a client side and a body part that is a model of a server side and learning is performed with a distributed learning method without directly sharing data.

3. The transformation of claim 1, wherein the preparing and the passing and then transmitting comprises randomly shuffling, using the permutation module, patch permutation before transmitting patch features from a client side to the server and transmitting the same.

4. The transformation method of claim 1, wherein the permutation module is a random patch permutation module and configured to randomly patch-permutate data transmitted from a client side to a server side to transmit representational feature data in which original data is unidentifiable.

5. The transformation method of claim 1, wherein the permutation module is a random patch permutation module and configured to allow only some of model weights aggregated and distributed by a server side to be shared to make it infeasible to restore the entire data in reverse order.

6. The transformation method of claim 1, further comprising:
performing, by the body part of the vision transformer model of the server, forward pass with permutated patch features and transmitting encoded features back to the client.

7. The transformation method of claim 6, further comprising:
reversing, by the client, permutation with a stored key, transmitting reverted features to a task-specific tail part, and yielding a final output,
wherein the preparing and the passing and then transmitting comprises randomly shuffling, using the permutation module, patch permutation before transmitting patch features from a client side to the server and storing the key to reverse the permutation on the client side.

8. The transformation method of claim 6, wherein the transmitting the encoded features back to the client comprises performing, using the permutation module, back-propagation in order of tail, body, and head of the vision transformer model that is the opposite way of forward propagation.

9. A transformation method using a distributed learning-based multi-task vision transformer through random patch permutation, the transformation method comprising:
randomly shuffling, using a permutation module, patch permutation and storing a key to reverse permutation on a client side and then transmitting patch features from a client to a server;
performing, by a body part of a vision transformer model of the server, forward pass with permutated patch features and transmitting encoded features back to the client; and
reversing, by the client, the permutation with the stored key, passing reverted features to a task-specific tail part, and yielding a final output.

10. The transformation method of claim 9, wherein the transmitting the encoded features back to the client comprises performing, using the permutation module, back-propagation in order of tail, body, and head of the vision transformer model that is the opposite way of forward propagation.

11. The transformation method of claim 9, wherein the transmitting the patch features to the server comprises preparing, using a task non-specific patch embedder, a patch embedding for each client and passing the patch embedding through the permutation module and then transmitting the patch embedding to the server.

12. The transformation method of claim 11, further comprising:
storing, by the server, the received patch embedding and using the patch embedding to update body and tail parts of a vision transformer model.

13. A distributed learning-based multi-task vision transformer through random patch permutation, the transformer comprising:
a head part configured to prepare, using a task non-specific patch embedder, a patch embedding for each client, to pass the patch embedding through a permutation module and then transmit the patch embedding to a server; and
a feature storage configured to store received patch embedding in the server and to use the patch embedding to update and tail parts of a vision transformer model.

14. The transformer of claim 13, wherein the vision transformer model that performs multi-task learning is separated into a head and tail part a model of a client side and a body part that is a model of a server side and learning is performed with a distributed learning method without directly sharing data.

15. The transformer of claim 13, wherein the head part is configured to randomly shuffle, using the permutation module, patch permutation before transmitting patch features from a client side to the server and to transmit the same.

16. The transformer of claim 13, wherein the permutation module is a random patch permutation module and configured to randomly patch-permutate data transmitted from a client side to the server side to transmit representational feature data in which original data is unidentifiable.

17. The transformer of claim 13, wherein the permutation module is a random patch permutation module and configured to allow only some of model weights aggregated and distributed by the server side to be shared to make it infeasible to restore the entire data in reverse order.

18. The transformer of claim 13, further comprising:
a body part configured to perform forward pass with permutated patch features in the body part of the vision transformer model of the server and to transmit encoded features back to the client.

19. The transformer of claim 18, further comprising:
a tail part configured to reverse, by the client, permutation with a stored key, to transmit reverted features to a task-specific tail part, and to yield a final output,
wherein the head part is configured to randomly shuffle, using the permutation module, patch permutation before transmitting patch features from a client side to the server and to store the key to reverse the permutation on the client side.

20. The transformer of claim 18, wherein the body part is configured to perform, using the permutation module, back-propagation in order of tail, body, and head of the vision transformer model that is the opposite way of forward propagation.
